(19) United States
(12) Patent Application Publication
(10) Pub. No.: US 2017/0239796 A1
(43) Pub. Date: Aug. 24, 2017

(54) Electronic Torque Wrench With Identification System

(71) Applicant: Gauthier Biomedical, Inc., Grafton, WI (US)

(72) Inventors: Michael T. Gauthier, Grafton, WI (US); Austin A.S. Braganza, Grafton, WI (US)

(21) Appl. No.: 15/429,452

(22) Filed: Feb. 10, 2017

Related U.S. Application Data
(60) Provisional application No. 62/297,278, filed on Feb. 19, 2016.

(51) Int. Cl. B25B 23/142; A61B 90/90; H04B 5/00; B25B 21/00; G06K 7/10

(52) U.S. Cl. CPC B25B 23/1425; B25B 21/004; G06K 7/10366; H04B 5/0062; A61B 90/90; A61B 90/98

(57) ABSTRACT

A torque wrench is provided that includes a radio frequency identification (RFID) or near field communication (NFC) sensing mechanism that enables the wrench to receive information regarding the different attributes of a shaft that is operably connected to the wrench. The sensing mechanism can communicate with a corresponding RFID/NFC tag located in a shaft that is releasably connected to the wrench in order to receive information from the tag on the status of the shaft and set points for the application of torque using the shaft. Information can also be written and stored on the tag concerning the length of time the shaft has been in use.

### CROSS-REFERENCE TO RELATED APPLICATIONS

The present application claims priority from U.S. Provisional patent Application Ser. No. 62/297,278, filed on Feb. 19, 2016, the entirety of which is expressly incorporated by reference herein for all purposes.

### FIELD OF THE INVENTION

The present invention relates generally to wrenches, and more specifically to an electronic torque wrench.

### BACKGROUND OF THE INVENTION

Torque wrenches are utilized to drive fasteners into a substrate while allowing the user to control the amount of force exerted on the fastener, such as to avoid damage to the substrate.

In certain medical procedures, different implants are positioned within the patient and secured therein by fasteners driven by the tools using fasteners connected to the tool. The forces exerted on the fasteners should not exceed predetermined levels in order to avoid damage from being done to the patient or to the implant/fastener. However, without suitable feedback from the wrench in the form of alerts or displays it is difficult for the user to know the proper level of force or torque to or being applied the fastener.

Prior art tools and wrenches enable user to input various set or alarm points into the tool that provide this feedback to the user for the particular fasteners and corresponding shafts used with the fasteners. This enables the user to be notified by the tool when a preset limit for the torque to be applied to a fastener is close to or has been reached by the user. This allows the user to properly tighten the fasteners in a manner that significantly limits the potential for over- or under tightening of the fastener, thereby reducing complications with regard to the medical procedure in which the implant and fasteners are being utilized.

However, with the wide variety of shafts and fasteners utilized with any given tool, the torque of other forces applied by the tool to the fastener can vary significantly, such as when a manual driving tool is utilized versus a motorized driving tool. Also, the different types of fasteners can require different forces to engage the fasteners to similar depth based on the construction of the fasteners. As such, in order to compensate for the differences between the tools, it is necessary to manually input and/or reprogram the alerts into the tool to correspond to the particular fastener and corresponding shaft connected to the tool/wrench to drive the fastener.

Thus, it is desirable to develop a driving tool such as a torque wrench, that is capable of being set without the need for manually inputting the alarm limits and set points for the particular fasteners and shafts.

### SUMMARY OF THE INVENTION

Briefly described, according to one exemplary and non-limiting embodiment of the invention a torque wrench includes a radio frequency identification (RFID) or near field communication (NFC) sensing mechanism or tag that enables the wrench to receive information regarding the different attributes of a shaft that is operably connected to the wrench. The sensing mechanism can communicate with a corresponding tag located on or in a shaft that is connected to the wrench. The tag stores information on the shaft and its manufacturer, usage and configuration and the fasteners, as well as any other relevant information concerning the shaft. This information can be transmitted to the wrench upon connection of the shaft to the wrench and used by a control system within the wrench to provide the user with alarm and set point indications during the use of the wrench and shaft to tighten one or more fasteners.

According to another exemplary embodiment of the invention, the sensing mechanism can be utilized to record information about the shaft connected to the tool, and to store that information on the shaft. For example, the information written to the tag disposed on the shaft can include, but is not limited to, the amount of time a shaft has been in use, in order to prevent a shaft from being used past its useful life and to prevent unauthorized or counterfeit shafts from being able to be utilized with the wrench.

Numerous other aspects, features, and advantages of the invention will be made apparent from the following detailed description together with the drawings figures.

### BRIEF DESCRIPTION OF THE DRAWINGS

The drawings illustrate the best mode currently contemplated of practicing the present invention.

In the drawings:

FIG. 1 is an isometric view of an electronic driving tool according to an exemplary embodiment of the invention;

FIG. 2 is a cross-sectional view along line 2-2 of FIG. 1; and

FIG. 3 is a cross-sectional view of another exemplary embodiment of the invention.

### DETAILED DESCRIPTION OF THE INVENTION

Reference will now be made in detail to various embodiments of the invention, one or more examples of which are illustrated in the accompanying drawings. Each example is provided by way of explanation, not limitation, of the invention. In fact, it will be apparent to those skilled in the art that modifications and variations can be made in the present invention without departing from the scope and spirit thereof. For instance, features illustrated or described as part of one embodiment may be used on another embodiment to yield a still further embodiment. Thus, it is intended that the present invention covers such modifications and variations as come within the scope of the appended claims and their equivalents.

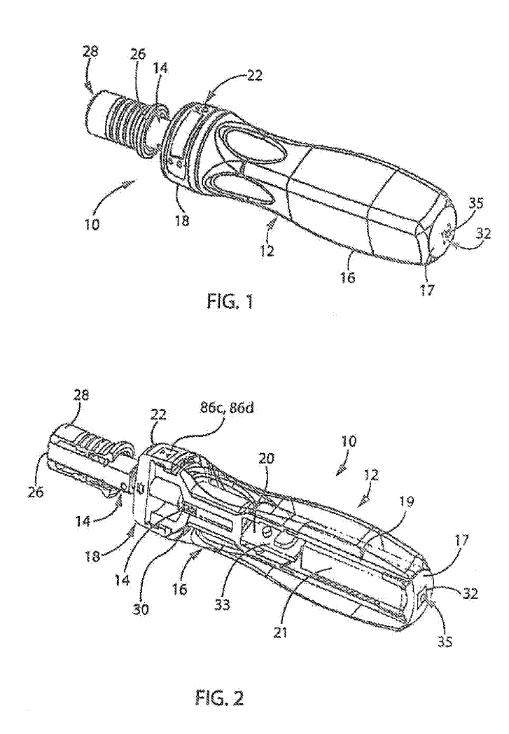

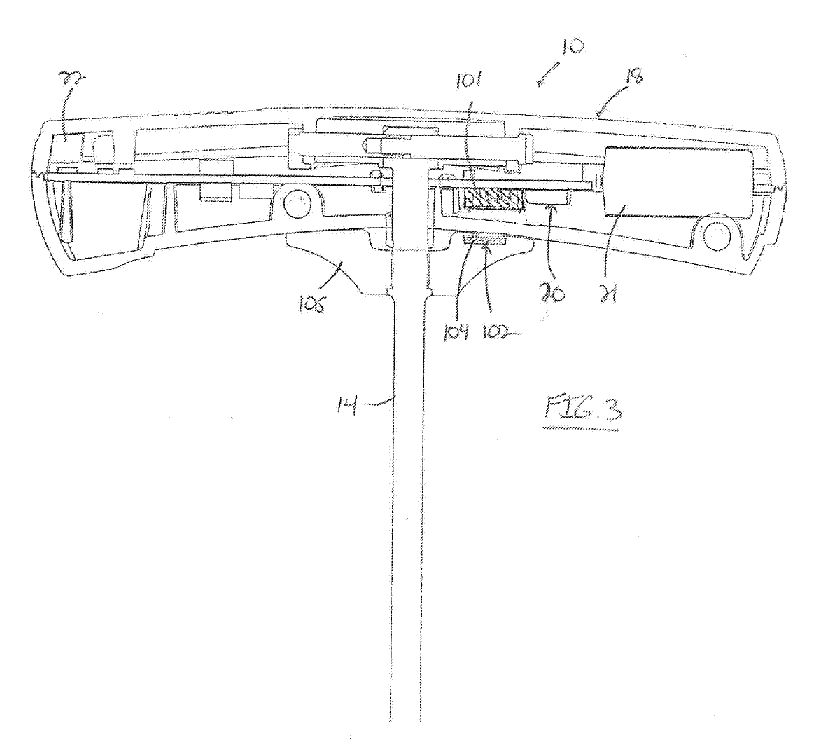

Referring now to FIGS. 1 and 2, a driving tool or device 10, such as an in-line electronic torque wrench, is illustrated in accordance with one exemplary and non-limiting embodiment of the invention, such as that disclosed in U.S. Pat. No. 9,358,672, entitled Electronic Torque Wrench, the entirety of which is expressly incorporated herein by reference for all purposes. The electronic torque wrench 10 includes a wrench body 12, a ratchet/wrench shaft 14, a resilient grip handle 16, a housing 18, a battery assembly 19, and an electronics unit 20 with a user interface or display 22 including display control buttons 86c, 86d. In the illustrated exemplary embodiment, wrench body 12 is of generally a tubular construction, and receives shaft 14 at a first end and a power supply or battery assembly 19 at a second end, secured therein by an end cap 17. Housing 18 is mounted therebetween and carries electronics unit 20.

As shown, a front end 26 of shaft 14 includes an adapter 28 connected to a suitable ratcheting mechanism (not shown) such as that shown in U.S. Pat. No. 7,413,065, incorporated by reference herein in its entirety for all purposes. The adapter 28 is configured to receive variously sized sockets, extensions, etc., that are releasably engaged within the adapter 28. The adapter 28 can also be detachably connected to the shaft 14 by any suitable mechanism.

The attachment mechanism for securing the shaft 14 to the body 12 includes a sensor 30 configured to sense the torque or strain exerted by the wrench 10 through the shaft 14 onto a fastener (not shown). The sensor 30 can take any of a number of suitable forms, such as a strain gauge, a Hall sensor, and a piezoelectric sensor, among others.

The sensor 30 is operably connected to the electronics unit 20, such that the signal generated by the sensor 30 can be transmitted to the unit 20. Once in the unit 20, the unit 20 can utilize the signal for a variety of purposes, such as to calculate a torque value from the signal to provide a real-time indication of the torque applied via the wrench 10 on the display 22.

The operation of the electronics unit 20 and the sensor 30 is controlled by a switch 32 disposed on the body 12, and operably connected between the unit 20 and sensor 30, and the power supply 19, e.g., batter 21, at the opposite end. Thus, depressing the protruding portion 35 of the switch 32 enables power to be selectively applied from the power supply 19 to the unit 20 and sensor 30 as desired. The engagement of the housing 18, the body 12 the handle 16 and the end cap 17 provide an enclosure or barrier 33, which can also be formed separately, for the battery assembly 19, the electronics unit 20 and the sensor(s) 30 that enables the wrench 10 to be autoclaved for sterilization purposes without damaging the battery assembly 19, the electronics unit 20 or the sensor 30, as well as the display 22.

Looking now at FIGS. 2 and 3, where FIG. 3 illustrated another exemplary and non-limiting embodiment of the invention where the tool 10 takes the form of a T-handle wrench, the wrench 10 further includes an RFID/NFC sensing mechanism 100 disposed within the housing 18. The mechanism 100 is formed with a reader 101 operably connected to the electronics unit 20 and display 22. The reader 101 is a device capable of sending and receiving information from a suitable RFID/NFC chip or tag 102 when the tag 102 is placed within a certain proximity of the reader 101. The reader 101 can operated in a passive manner where the tag 102 is independently powered, such that the tag 102 actively sends a signal that is received by the reader 101, and/or in an active manner where the reader 101 sends out a querying signal that serves to both to operate and interrogate the tag 102.

In either mode of operation, the reader 101 and is capable of sensing and receiving information from an RFID/NFC chip or tag 102. In the illustrated exemplary embodiment of FIG. 2, the tag 102 is disposed on or operatively connected to the shaft 14. The chip 102 includes a suitable storage medium 104 in which information about the shaft 14, is stored. This information can include, but is not limited to, information on the manufacturer of the shaft 14, the amount of time the shaft 14 has been in use, and the set points/torque limits for the use of the shaft 14 in tightening a fastener using the tool 10 and shaft 14. When the tool 10 is in use, the reader 101 can update the information contained in the storage medium 104 on the tag 102, thereby providing information on the length of use of the tool 10 and shaft 14. Each time the tag 102 is accessed by the reader 101, the information obtained from the tag 102 can be illustrated on the display 22 or in any other suitable manner to illustrate the current condition of the shaft 14 and whether the shaft 14 has exceeded its useful life, for example. Further, the tag 102 can supply information relating to the maximum torque to be applied using the shaft 14 and/or the maximum torque to be applied when engaging fasteners (not shown) of various types, with this information transmitted to the electronics control unit 20 from the reader 101 to automatically determine the torque set points for the procedure in which the tool 10 is going to be used.

Looking now at FIG. 3, in another exemplary and non-limiting embodiment, the shaft 14 is illustrated as being detachable from the housing 18, such that different shafts 14 suitable for different fasteners (not shown) or operations can be engaged with the housing 18. In this exemplary embodiment, while the tag 102 can be disposed on the shaft 14 in any position where the reader 101 can sense and communicate with the tag 102, the tag 102 is disposed within a hub 106 disposed on the shaft 14. The hub 106 provides a locating feature to the shaft 14 and properly positions the shaft 14 adjacent the housing 18 when the shaft 14 is secured to the housing 18. In this position, the tag 102 located within the hub 106 is positioned near the reader 101 such that the information stored on the tag 102 can be transferred to the reader 101 and electronics unit 20. With that information the electronics unit 20 can operate the display 22 during use of the tool 10 to indicate to the user when the force/torque applied by the wrench to a fastener has reached one or more predetermined set points or exceeded the limit for the use of the shaft 14 and/or fastener. In addition, the information stored on and/or supplied by the tag 102 can provide an indication to the user of the length of service time for the shaft 14, thereby providing an indication to the user of whether the shaft 14 can continue to be used in procedures, or if the shaft 14 requires replacement. Further, the information retained ion the tag 102 and accessed by the reader 101 can include compatibility information concerning those tools 10 with which the shaft 14 can be utilized. In this manner, the use of a shaft 14 that is incompatible with a particular tool 10 can be avoided prior to the shaft 14 being utilized in a procedure.

In this manner, the electronics unit 20 can be configured for use with a particular shaft 14 simply by the connection of the hub 106 to the housing 18, enabling the tag 102 to be sensed and read by the reader 101 with logic programmed in the device 10 transmitted to and utilized by the electronics unit 20 to set the appropriate torque alert level for that shaft 14. The wrench 10 can correspondingly be set with multiple set points automatically without the need for any manual configuration, thereby the need for manual input and its chances for user error.

In alternative exemplary embodiments, the tag reader 101 can be configured as a part of the electronics unit 20, and the tag 102 can be an active or a passive tag embedded in the shaft 14 of an instruments or in the implant on a surgical tray. When the tag 102 is present in a location on the shaft 14 other than the hub 106, the housing 18 and reader 101 can be positioned in close proximity to the tag 102 on the shaft 14 in order for the information to be sensed and received by the reader 101 and electronics unit 20 prior to engagement of the shaft 14 to the housing 18 for use.

Various other embodiments of the invention are contemplated as being within the scope of the filed claims particularly pointing out and distinctly claiming the subject matter regarded as the invention.

1. A driving tool comprising:
a) a body capable of being subjected to temperatures and pressures associated with medical sterilization procedures;
b) a shaft operably connected to the body and engageable with a fastener; and
c) a housing including an RFID/NFC sensing mechanism operably connected to the electronics unit and operable to sense and receive information from an RFID/NFC tag adapted to be positioned adjacent the reader.

2. The tool of claim 1 wherein the tag is positioned on the shaft.

3. The tool of claim 1 wherein the tag is disposed within a hub on shaft and releasably engagable with the housing.

4. The tool of claim 1 wherein the tag includes an electronic storage medium on which information about torque set points are stored.

5. The tool of claim 1 wherein the tag includes an electronic storage medium on which information on the length of time the shaft has been in operation is stored.

6. The tool of claim 1 wherein the tag includes an electronic storage medium on which information about driving tools compatible with the shaft is stored.

7. A method of positioning a fastener with respect to a substrate comprising the steps of:
a) providing the tool of claim 1;
b) engaging a shaft with the tool; and
c) operating the tool to position the fastener within the substrate.

8. The method of claim 7 wherein the step of providing the tool comprises engaging the shaft with the body to position the RFID/NFC sensing mechanism adjacent the RFID/NFC tag.

9. The method of claim 7 wherein the step of providing the tool comprises positioning the shaft relative to the body to position the RFID/NFC sensing mechanism adjacent the RFID/NFC tag.

10. The method of claim 7 further comprising the step of accessing information located on an electronic storage medium on the tag.

11. The method of claim 10 wherein the step of accessing the information comprises accessing torque set point data.

12. The method of claim 10 wherein the step of accessing the information comprises accessing shaft use data.

13. The method of claim 10 wherein the step of accessing the information comprises accessing compatible tool data.

14. The method of claim 10 further comprising the step of writing information to an electronic storage medium on the tag.

15. The method of claim 14 wherein the step of writing the information comprises storing shaft use data on the electronic storage medium.
